(19) United States
(12) Patent Application Publication
(10) Pub. No.: US 2013/0225967 A1
(43) Pub. Date: Aug. 29, 2013

(54) SMALL WIRELESS PORTABLE EKG SYSTEM

(71) Applicant: Anthony Esposito, Scottsdale, AZ (US)

(72) Inventor: Anthony Esposito, Scottsdale, AZ (US)

(21) Appl. No.: 13/778,011

(22) Filed: Feb. 26, 2013

Related U.S. Application Data
(60) Provisional application No. 61/605,128, filed on Feb. 29, 2012.

(51) Int. Cl. A61B 5/0404; A61B 5/044; A61B 5/00; A61B 5/0408

(52) U.S. Cl. CPC A61B 5/0404; A61B 5/04087; A61B 5/044; A61B 5/0006
USPC 600/392; 600/509

(57) ABSTRACT

A wallet-card sized, flexible, largely self-contained electrocardiogram (EKG) device able to wirelessly communicate EKG data to a smart phone, or similarly functional device, hosting an application that can receive and display the EKG data, as well as do some analysis to identify particular heart conditions and display that information, or data product. The EKG device processes data from its integral electrodes into EKG data, can store up to an hour of data, and can wirelessly transmit the EKG data from storage or from real-time processing. The smart phone receives the EKG data, makes it available to the application, and displays results produced by the application, which may include alarms or identification of specific heart problems. The smart phone can further communicate the EKG data and additional data products to a web server application, which can perform additional analysis for display, or communicate to a medical provider.

### RELATED APPLICATIONS

This application claims the benefit of U.S. provisional patent application Ser. No. 61/604,128 filed Feb. 28, 2012 naming the same inventor.

### TECHNICAL FIELD

This invention relates to EKG devices for providing electrocardiography. More particularly, it relates to wireless, portable, flexible EKG devices that are about the size of a credit card.

### BACKGROUND

EKG devices provide information about electrical activity in the thorax, which provides medical information about the heart. EKG devices are used, for instance, for diagnosing heart attacks. Some EKG devices are bulky and have up to twelve electrical leads that must be attached to the patient which requires a significant amount of time, even when little time is available. Much of the bulk is associated with recording and printing of the electrocardiogram for visual analysis by a specialist.

Therefore, a need exists for small portable EKG device that can be quickly attached to the patient and can rapidly provide information about the patient's heart.

### OBJECTS AND FEATURES OF THE INVENTION

A primary object and feature of the present invention is to overcome the above-mentioned problems and fulfill the above-mentioned needs.

Another object and feature of the present invention is to provide an EKG device that is about the size of a credit card. Another object and feature of the present invention is to provide an EKG device that wirelessly transmits data to a computer or smart phone for display and analysis. Another object and feature of the present invention is to provide an EKG device that is disposable. Another object and feature of the present invention is to provide an EKG device that can be quickly connected to the patient. Another object and feature of the present invention is to provide an EKG device that sends data to a computer application that can analyze the data and provide alarms for particular heart conditions. Another object and feature of the present invention is to provide an EKG application for computer or smart phone that can contact the patient's doctor and relay the electrocardiogram to that doctor. Another object and feature of the present invention is to provide an EKG application for computer or smart phone that can relay the electrocardiogram produced by the EKG device to a website for further analysis. It is a further object and feature of the present invention to provide an EKG device that can be reconditioned and re-used. It is a further object and feature of the present invention to provide an EKG device that can store up to an hour of data. Another object and feature of the present invention is to provide an EKG device that can adhere to the patient's chest.

It is an additional primary object and feature of the present invention to provide such a system that is efficient, inexpensive and handy. Other objects and features of this invention will become apparent with reference to the following descriptions.

### SUMMARY OF THE INVENTION

In accordance with a preferred embodiment hereof, this invention provides a small wireless portable EKG device and a computer application, which may be a smart phone application, together comprising the EKG system. The EKG device is a credit-card sized (or smaller) device with an adhesive backing, covered by a release layer until used, at least two conductive electrodes communicatively coupled to a signal processor which processes the raw data into an electrocardiogram. The signal processor is communicatively coupled to a processor memory, where the electrocardiogram data may be stored. The processor memory and the signal processor are communicatively coupled to a wireless transceiver which, in turn, is communicatively and wirelessly coupled, during at least a portion of operation, to a computer, smart phone, or the like. The computer hosts an application for at least receiving and displaying the electrocardiogram, and optionally for contacting a cardiologist, sending the data to another computer for further analysis, or further analyzing the data in the application.

The present invention provides a small wireless portable electrocardiogram (EKG) system including; a small, flexible, wireless, and portable EKG device; and a first computer application able to receive wireless data originally sent from the EKG device and to provide for display at least one of the data and at least one data product. The system, where the EKG device is either a flexible panel no larger than two inches wide by three inches long by fifteen hundredths of an inch thick or a flexible panel no more than two inches wide and fifteen hundredths of an inch thick and having a length of at least three inches that may be rolled up for storage or transport. The system, where the EKG device includes: a flexible substrate having first and second sides and first and second ends; a user-activated battery-based power supply mounted on the second side of the substrate and coupled to power-consuming elements of the EKG device; a plurality of flexible electrodes separated into first and second groups each proximate respective first and second ends on the first side of the substrate; an electrically conductive adhesive surface surrounding each electrode of the plurality of electrodes on the first side of the substrate; an electrically non-conductive adhesive surface surrounding each electrically conductive adhesive surface on the first side of the substrate; a release layer covering the adhesive surfaces prior to use; and a signal processor, formed of flexible circuitry, positioned underneath the electrically non-conductive adhesive surface and in electronic communication with each electrode of the plurality of electrodes. The system, where the EKG device further includes: a processor memory, formed of flexible circuitry, positioned underneath the electrically non-conductive adhesive surface and in electronic communication with the signal processor; a wireless transceiver, formed of flexible circuitry, positioned underneath the electrically non-conductive adhesive surface and in electronic communication with the signal processor and with the processor memory; and where the wireless transceiver is able to receive commands to transmit one of: real-time data from the signal processor; and stored data from the processor memory. The system, where the first computer application is hosted on a device that includes a device able to: receive data from via a wireless link; host the computer application that is capable of creating at least one data product; and display the data and the at least one data product; The system, where the device hosting the first computer application is hosted on a device that is able to communicate, at least partially over a wireless link, with a website server. The system, further including a second computer application hosted on a website server able to: communicate, at least partially over a wireless link, with a computer hosting the computer application; host the a second computer application able to: analyze the data; create at least one data product; format the data for display; store the data and the at least one data product; and provide the data and the at least one data product for display. The system, where the website server is further able to communicate the data and a data product to a medical provider for analysis.

The present invention further provides a small wireless portable EKG system including; a small, wireless, flexible, and portable EKG device; a first computer application, hosted on a portable device, able to receive wireless data originally sent from the EKG device and to provide the data and/or a data product for display; and a second computer application able to receive wireless data and at least one data product originally sent from the EKG device and to provide the data, a data product, and an additional data product for display. The system, where the EKG device includes: a substrate having first and second sides and first and second ends and further includes one of a wallet card and a roll; a user-activated battery-based power supply, including one or more Zinc-air batteries, mounted on the second side of the substrate and coupled to power-consuming elements of the EKG device; a plurality of electrodes separated into first and second groups each proximate respective first and second ends on the first side of the substrate; an electrically conductive adhesive surface surrounding each electrode of the plurality of electrodes on the first side of the substrate; an electrically non-conductive adhesive surface surrounding each electrically conductive adhesive surface on the first side of the substrate; a release layer covering the adhesive surfaces prior to use; and a signal processor, formed of flexible circuitry, positioned underneath the electrically non-conductive adhesive surface and in electronic communication with each electrode of the plurality of electrodes and powered by the power supply. The system, where the EKG device further includes: a processor memory, formed of flexible circuitry, positioned underneath the electrically non-conductive adhesive surface and in electronic communication with the signal processor; and a wireless transceiver, formed of flexible circuitry, positioned underneath the electrically non-conductive adhesive surface and in electronic communication with the signal processor and with the processor memory. The system, where the wireless transceiver is able to receive commands to transmit either real-time data from the signal processor or stored data from the processor memory. The system, where the first computer application is hosted on a device that is able to: receive data from via a wireless link; host the computer application that is capable of creating at least one data product; and display the data and a data product. The system, where the first computer application is hosted on a device that is able to communicate with a website server at least partially over a wireless link. The system, further including the second computer application hosted on a website server, the website server able to: communicate, at least partially over a wireless link, with the first computer hosting the first computer application; use the second computer application to: analyze the data; create at least one additional data product using the second computer application; format the data for display; and provide the data and the data product for display; and store the data, the data product, and the additional data product. The system, where the website server is further able to communicate to a medical provider.

The invention further provides a small wireless portable EKG system including; a small, flexible, wireless, and portable EKG device; a first computer application, hosted on a portable device, able to receive wireless data originally sent from the EKG device and to provide the data and a data product for display; and a second computer application, hosted on a web server, able to receive wireless data and the data product originally sent from the EKG device and to provide the data, the data product, and an additional data product for display. The system, where the EKG device includes: a flexible substrate having first and second sides and first and second ends and includes one of a wallet-sized card and a roll; a user-activated battery-based power supply mounted on the second side of the substrate and including one or more Zinc-air batteries; first and second electrodes proximate respective first and second ends on the first side of the substrate; first and second electrically conductive adhesive surfaces surrounding respective first and second electrodes on the first side of the substrate; an electrically non-conductive adhesive surface between the first and second electrically conductive adhesive surfaces on the first side of the substrate; a release layer covering the adhesive surfaces prior to use; a signal processor, formed of flexible circuitry, positioned underneath the electrically non-conductive adhesive surface and in electronic communication with the first and second electrodes; a processor memory, formed of flexible circuitry, positioned underneath the electrically non-conductive adhesive surface and in electronic communication with the signal processor; and a wireless transceiver, formed of flexible circuitry, positioned underneath the electrically non-conductive adhesive surface and in electronic communication with the signal processor and with the processor memory; where the wireless transceiver is able to receive commands to transmit either real-time data from the signal processor or stored data from the processor memory. The system, where the first computer application is hosted on the portable device that is able to: receive data from the EKG device via a wireless link; host the computer application that is capable of creating a data product; display the data and the data product; accept, store, and communicate user input; and communicate with a website server at least partially over a wireless link. The system, where the web server is able to: communicate, at least partially over a wireless link, with the portable device hosting the first computer application; host the second computer application that is able to: analyze the data; create an additional data product; format the data and the additional data product for display; store the data, the data product, and the additional data product; provide the data, the data product ,and the additional data product for display; and communicate the data, the data product, and the additional data product to a medical provider for analysis.

### BRIEF DESCRIPTION OF THE DRAWINGS

The above and other objects and advantages of the present invention will become more apparent from the following description taken in conjunction with the following drawings in which:

FIG. 1A is a diagrammatic front perspective view illustrating an exemplary EKG device, according to a preferred embodiment of the present invention;

FIG. 1B is a diagrammatic rear perspective view illustrating an exemplary EKG device of FIG. 1A, according to a preferred embodiment of the present invention;

FIG. 2A is a diagrammatic front plan view illustrating the exemplary EKG device of FIG. 1A with the release layer removed, according to a preferred embodiment of the present invention;

FIG. 2B is a diagrammatic plan view illustrating the exemplary EKG device of FIG. 1A with the release layer removed, according to a preferred embodiment of the present invention; and

FIG. 3 is a view illustrating an exemplary EKG system incorporating the EKG device of FIG. 1, according to a preferred embodiment of the present invention.

### DETAILED DESCRIPTION OF THE INVENTION

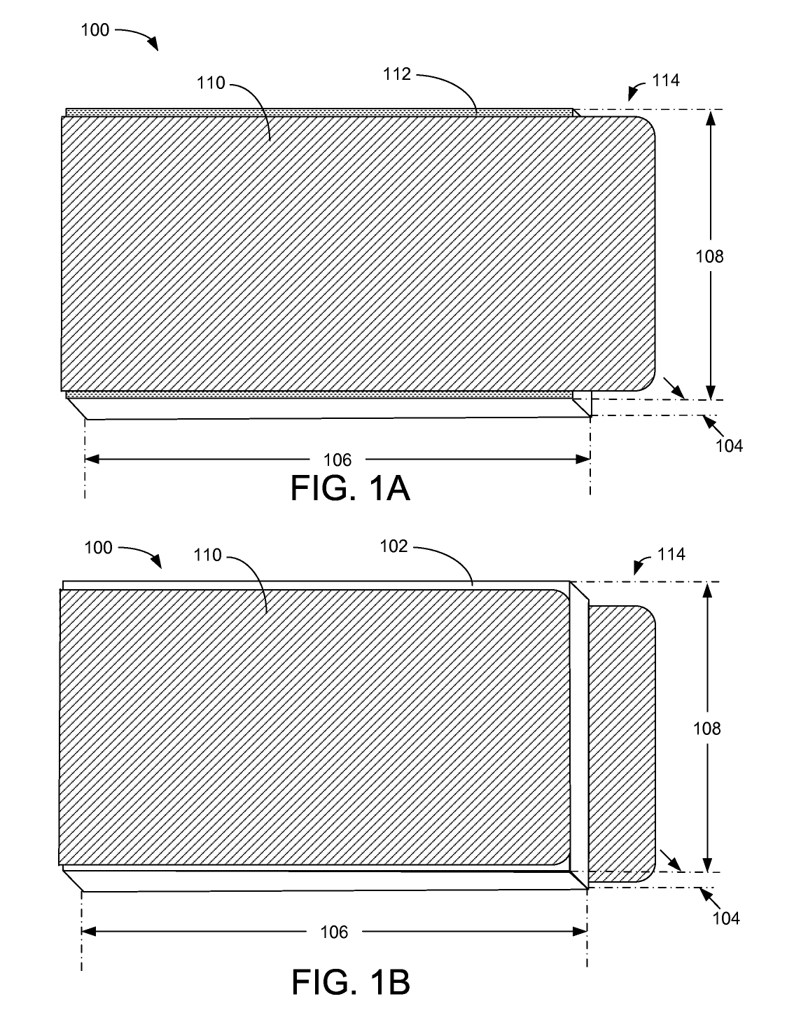

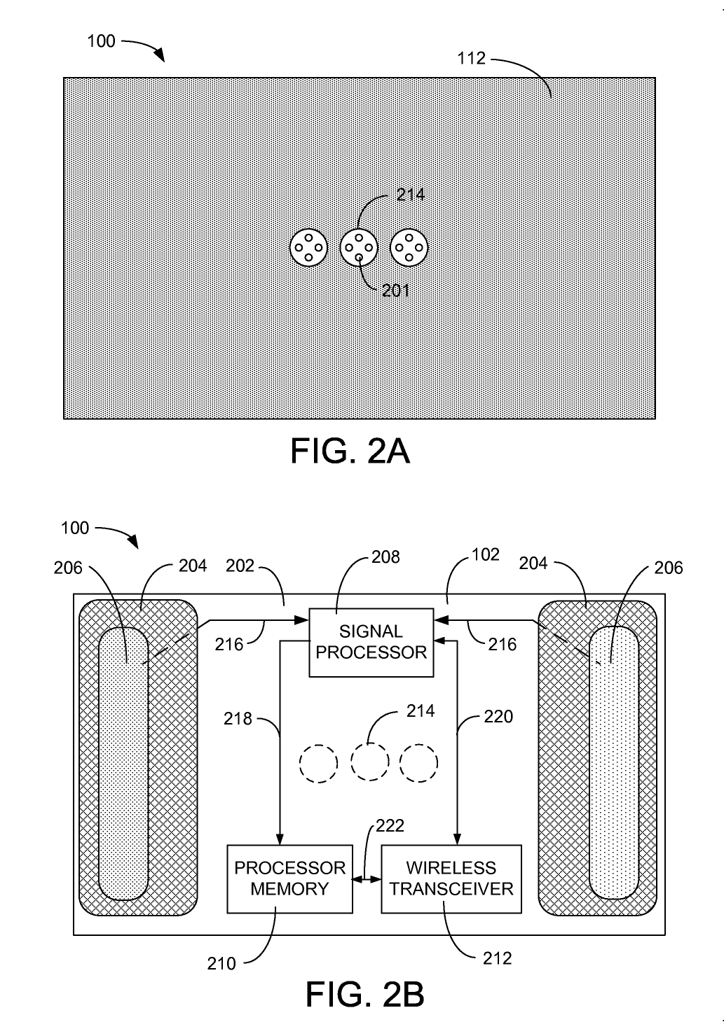

FIG. 1A is a diagrammatic front perspective view illustrating an exemplary EKG device 100, according to a preferred embodiment of the present invention. Release layer 110 covers at least a battery-bearing portion (see FIG. 2A) of flexible non-conductive substrate 112. EKG device 100 includes the flexible non-conductive substrate 112 and a removable wrap-around release layer 110 which, when removed, exposes Zinc-air batteries 2144 (See FIG. 2A) to the atmosphere, thereby activating the batteries 214. Release layer 110 is typically used once, and then discarded after removal. In an alternate embodiment, wireless electrode patch 102 may be refurbished after use, and either a new release layer 110 may be provided or the original may be reused. EKG device 100 preferably has a thickness 104 no greater than fifteen hundredths of an inch, a length 106, without tab 114, of no greater than three inches, and a width 108 of no greater than two inches. In various additional embodiments, these size limits may be exceeded. The dimensions 106 and 108 are preferably as small as possible consistent with spacing the electrodes 206 (See FIG. 2B) far enough apart to get useful data. The thickness 14 is preferably small enough to allow flexibility and easy carriage, such as in a wallet. Tab 114 extends beyond the length 106 to enable the user to easily grasp the release layer 110 for removal.

FIG. 1B is a diagrammatic front perspective view illustrating an exemplary EKG device 100 of FIG. 1A, according to a preferred embodiment of the present invention. EKG device 100 includes the adhesively attachable wireless electrode patch 102 and the removable wrap-around release layer 110 which, when removed, exposes an adhesive surface for attaching the wireless electrode patch 102 to a patient.

FIG. 2A is a diagrammatic front plan view illustrating the exemplary EKG device 100 of FIG. 1A with the release layer 110 removed, according to a preferred embodiment of the present invention. Batteries 214 (one of three labeled) are mounted on the flexible non-conductive substrate 112 and are activated when release layer 110 is removed. Removal of release layer 110 is done manually by the user. The first action of removing the release layer from the front of substrate 112 uncovers air holes 201 (one of twelve labeled) on the Zinc-air batteries 214, allowing air to enter the Zinc-air battery and thereby activate it. The flexible electrical conductors from the batteries 214 to the power-consuming elements of the EKG device 100 are not shown for simplicity of the drawing, but are well known to those of ordinary skill in the art.

FIG. 2B is a diagrammatic plan view illustrating an exemplary EKG device 100 of FIG. 1A with the release layer 110 removed, according to a preferred embodiment of the present invention. Wireless electrode patch 102 has the flexible non-conductive substrate 112 supporting an electrically non-conductive adhesive 202 and electrically conductive adhesive 204 surrounding metallic foil electrodes 206, as shown. In operation, the metallic foil electrodes 206 are placed in contact with a patient's skin to pick up electrical signals generated by the heart. The surrounding conductive adhesive 206 maintains the contact between the metallic foil electrodes 206 and the patient's skin. Non-conductive adhesive 202 covers the remainder of the contact surface of the wireless electrode patch 102 to maintain contact with the patient, without shorting the electrodes 206 to one another.

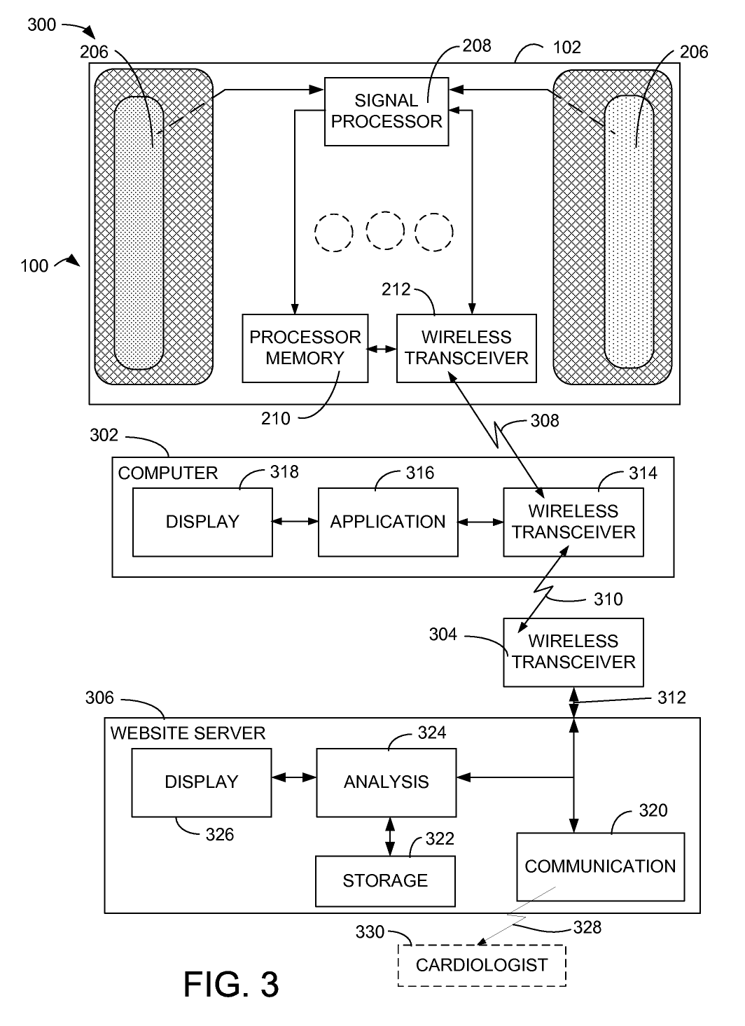

Behind the adhesive layer 202 and 204, and optionally behind the substrate, a signal processor 208 made of flexible circuitry receives signals picked up from metallic foil electrodes 206 via flexible leads 216. Signal processor 208 processes the raw data from the metallic foil electrodes into an electrocardiogram (EKG), or data product, and stores the electrocardiogram in processor memory 210 via flexible link 218. Wireless transceiver 212, also made of flexible circuitry, receives the electrocardiogram from processor memory 210 via flexible link 222 or directly from signal processor 208, via flexible link 220, and wirelessly transmits the electrocardiogram or the raw data to a computer 302 (see FIG. 3). The choice of source 208 or 210 of the data and/or data product may be determined by commands received by the wireless transceiver 212. When EKG device 100 is refurbished, previous EKG data stored in processor memory 210 is erased.

Zinc-air batteries 214 (one of three labeled can supply power for more than an hour. The advantage of Zinc-air batteries 214 is that they have a very long shelf life as they are not activated until ready to be used. Power linkages to power-consuming elements of the EKG device from the batteries 214 are not shown, as they are well known and would complicate the drawing unnecessarily. In an embodiment meant for refurbishment instead of disposability, the Zinc-air batteries may be replaced during refurbishment. In an additional embodiment, other types of batteries may be used.

FIG. 3 is a view illustrating an exemplary EKG system 300 incorporating the EKG device 100 of FIG. 1, according to a preferred embodiment of the present invention. EKG system 300 includes the EKG device 100, shown with release layer 110 removed and wireless electrode patch 102 visible, a computer 302 with wireless capability and, optionally, analysis and communication applications on a website server 306 accessible through a website. Computer 302 may be a smart phone, a personal computer, a tablet, PDA, or other type of computer with wireless capability and processing and display capabilities. The wireless link 308 between the wireless electrode patch 102 and the computer 302 may be a Bluetooth, Wi-Fi, broadband, or similar link 308. In an additional embodiment, a hardwired link may serve as link 308. In the illustrated example, the computer 302 is a smart phone 302 with a wireless transceiver 314 and display 318 communicatively coupled through an application 316 that formats the electrocardiogram for display 318. Thus, a user with a smart phone 302 may attach the wireless electrode patch 102 to the patient and observe the heart's electrical activity on the user's smart phone 302. In commerce, the EKG devices 100 may be sold in a kit with a smart phone application 316 and, optionally, a computer or smart phone 302. Application 316 is part of EKG system 300 which includes the smart phone or computer 302 as well. Application 316 may provide means for associating unique information for identifying the patient with the electrocardiogram. For example, the user may have an opportunity to enter the patient's name or patient's ID number into the application 316 using user input features available on the computer or smart phone 302. Further, information identifying the patient's medical provider, such as a cardiologist or other medical professional, may be entered to be used to route the electrocardiogram data to that medical provider.

Computer 302 may further communicate wirelessly with a website on a server 306 to supply the electrocardiogram data for further analysis and/or communication. Website server 306 is coupled to a wireless receiver 304 which receives signals from the wireless transceiver 314 of the computer 302 via wireless link 310 and supplies those signals to website server 306. The server 306 hosts applications for communication 320 and analysis 324, as well as providing intrinsic display 326 and storage 322 capability. In the illustrated example, applications for communication 320 and analysis 324 are part of EKG system 300. The analysis application 324 analyzes the electrocardiogram data and may provide alerts to the user for problematic data, such as that indicating a heart attack or other heart condition. In a preferred additional embodiment, the analysis application 324 may be hosted on smart phone 302 instead of or in addition to server 306. The communication application 320 responds to either electrocardiogram data or to alerts generated by the analysis application 324 to automatically dial the patient's cardiologist 330 via link 328, which may be wireless in whole or in part. Further, the communication application 320 may transfer the electrocardiogram data to cardiologist 330. In an additional preferred embodiment, the communication application 320 may be hosted on smart phone 302 instead of or in addition to server 306. In another additional preferred embodiment, wireless transceiver 212 may communicate directly with website server 306, such as in a hospital environment.

In exemplary operation, the user of EKG system 300 has in his possession one or more EKG devices 100 and a smart phone 302 with EKG application 316. The user removes the release layer 110 from the wireless electrode patch 102 and adheres the wireless electrode patch to the patient's chest. The user then selects the EKG application 316 on smartphone 302, and observes the patient's electrocardiogram. The user may remove himself from the immediate vicinity of the patient while observing the electrocardiogram, to the limit of the wireless range of the wireless transceiver 212 on electrode patch 102, to allow other care providers physical access to the patient.

In an alternate embodiment, more than two metallic electrodes 206 with surrounding electrically conducting adhesive 204 may be used. For non-limiting example, an EKG device may be a roll having six metallic electrodes 206 with surrounding electrically conducting adhesive 204 that is unrolled as a panel across the patient's chest as the release layer 110 is removed. The panel is preferably at least three inches long. In another non-limiting example, metallic electrodes 206 with surrounding electrically conducting adhesive 204 may be divided in half vertically to provide four electrodes, one at each corner of electrode patch 102, without any increase in size. Those of skill in the art, enlightened by the present disclosure, will recognize the wide variety of electrode configurations comprehended by the present invention.

The novel features of the invention include at least the following: small size; multiple electrodes 206 on a single electrode patch 102; wireless transceiver 212; internally powered 214 wireless transceiver 212 coupled to both processor memory 210 and signal processor 208 to provide either real-time or stored data; processing capability 208 within the electrode patch 102; speed of deployment; at least an hour of stored electrocardiogram data storable in processor memory 210 on the wireless electrode patch 102; the computer applications 316, 320, and 324; flexible electronic circuits 208, 210, 212, 216, 218, 220, and 220 on an electrode patch 102; providing alerts to particular heart conditions from an electrode patch 102; and alerting a cardiologist to the presence of electrocardiogram data and/or particular conditions identified in the data from the electrode patch 102.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing the exemplary embodiment or exemplary embodiments. It should be understood that various changes can be made in the function and arrangement of elements without departing from the scope of the invention.

I claim:

1. A small wireless portable electrocardiogram (EKG) system comprising;
a. a small, flexible, wireless, and portable EKG device; and
b. a first computer application operable to receive wireless data originally sent from said EKG device and to provide for display at least one of said data and at least one data product.

2. The system of claim 1, wherein said EKG device is one of:
a. a flexible panel no larger than two inches wide by three inches long by fifteen hundredths of an inch thick; and
b. a flexible panel no more than two inches wide and fifteen hundredths of an inch thick and having a length of at least three inches that may be rolled up for storage or transport.

3. The system of claim 1, wherein said EKG device comprises:
a. a flexible substrate having first and second sides and first and second ends;
b. a user-activated battery-based power supply mounted on said second side of said substrate and coupled to power-consuming elements of said EKG device;
c. a plurality of flexible electrodes separated into first and second groups each proximate respective said first and second ends on said first side of said substrate;
d. an electrically conductive adhesive surface surrounding each electrode of said plurality of electrodes on said first side of said substrate;
e. an electrically non-conductive adhesive surface surrounding each said electrically conductive adhesive surface on said first side of said substrate;
f. a release layer covering said adhesive surfaces prior to use; and
g. a signal processor, formed of flexible circuitry, positioned underneath said electrically non-conductive adhesive surface and in electronic communication with each electrode of said plurality of electrodes.

4. The system of claim 3, wherein said EKG device further comprises:
a. a processor memory, formed of flexible circuitry, positioned underneath said electrically non-conductive adhesive surface and in electronic communication with said signal processor;
b. a wireless transceiver, formed of flexible circuitry, positioned underneath said electrically non-conductive adhesive surface and in electronic communication with said signal processor and with said processor memory; and
c. wherein said wireless transceiver is operable to receive commands to transmit one of:
i. real-time data from said signal processor; and
ii. stored data from said processor memory.

5. The system of claim 1, wherein said first computer application is hosted on a device that comprises a device operable to:
a. receive data from via a wireless link;
b. host said computer application that is capable of creating at least one data product; and
c. display said data and said at least one data product.

6. The system of claim 1, wherein said device hosting said first computer application is hosted on a device that is operable to communicate, at least partially over a wireless link, with a website server.

7. The system of claim 1, further comprising a second computer application hosted on a website server operable to:
a. communicate, at least partially over a wireless link, with a computer hosting said computer application;
b. host said a second computer application operable to:
i. analyze said data;
ii. create at least one data product;
iii. format said data for display;
iv. store said data and said at least one data product; and
v. provide said data and said at least one data product for display.

8. The system of claim 7, wherein said website server is further operable to communicate said data and said at least one data product to a medical provider for analysis.

9. A small wireless portable EKG system comprising;
a. a small, wireless, flexible, and portable EKG device;
b. a first computer application, hosted on a portable device, operable to receive wireless data originally sent from said EKG device and to provide at least one of said data and at least one data product for display; and
c. a second computer application operable to receive wireless data and at least one data product originally sent from said EKG device and to provide said data, said at least one data product, and at least one additional data product for display.

10. The system of claim 9, wherein said EKG device comprises:
a. a substrate having first and second sides and first and second ends and further comprises one of a wallet card and a roll;
b. a user-activated battery-based power supply, comprising one or more Zinc-air batteries, mounted on said second side of said substrate and coupled to power-consuming elements of said EKG device;
c. a plurality of electrodes separated into first and second groups each proximate respective said first and second ends on said first side of said substrate;
d. an electrically conductive adhesive surface surrounding each electrode of said plurality of electrodes on said first side of said substrate;
e. an electrically non-conductive adhesive surface surrounding each said electrically conductive adhesive surface on said first side of said substrate;
f. a release layer covering said adhesive surfaces prior to use; and
g. a signal processor, formed of flexible circuitry, positioned underneath said electrically non-conductive adhesive surface and in electronic communication with each electrode of said plurality of electrodes and powered by said power supply.

11. The system of claim 10, wherein said EKG device further comprises:
a. a processor memory, formed of flexible circuitry, positioned underneath said electrically non-conductive adhesive surface and in electronic communication with said signal processor; and
b. a wireless transceiver, formed of flexible circuitry, positioned underneath said electrically non-conductive adhesive surface and in electronic communication with said signal processor and with said processor memory.

12. The system of claim 11, wherein said wireless transceiver is operable to receive commands to transmit one of:
a. real-time data from said signal processor; and
b. stored data from said processor memory.

13. The system of claim 9, wherein said first computer application is hosted on a device that is operable to:
a. receive data from via a wireless link;
b. host said computer application that is capable of creating at least one data product; and
c. display said data and said at least one data product.

14. The system of claim 9, wherein said first computer application is hosted on a device that is operable to communicate with a website server, at least partially over a wireless link.

15. The system of claim 9, further comprising said second computer application hosted on a website server, said website server operable to:
a. communicate, at least partially over a wireless link, with said first computer hosting said first computer application;
b. use said second computer application to:
i. analyze said data;
ii. create at least one additional data product using said second computer application;
iii. format said data for display; and
iv. provide said data and said at least one data product for display; and
c. store said data, said at least one data product, and said at least one additional data product.

16. The system of claim 7, wherein said website server is further operable to communicate to a medical provider.

17. A small wireless portable EKG system comprising;
a. a small, flexible, wireless, and portable EKG device;
b. a first computer application, hosted on a portable device, operable to receive wireless data originally sent from said EKG device and to provide said data and at least one data product for display; and
c. a second computer application, hosted on a web server, operable to receive wireless data and at least one data product originally sent from said EKG device and to provide said data, said at least one data product, and at least one additional data product for display.

18. The system of claim 17, wherein said EKG device comprises:
a. a flexible substrate having first and second sides and first and second ends and comprises one of a wallet-sized card and a roll;
b. a user-activated battery-based power supply mounted on said second side of said substrate and comprising one or more Zinc-air batteries;
c. first and second electrodes proximate respective said first and second ends on said first side of said substrate;
d. first and second electrically conductive adhesive surfaces surrounding respective first and second electrodes on said first side of said substrate;
e. an electrically non-conductive adhesive surface between said first and second electrically conductive adhesive surfaces on said first side of said substrate;
f. a release layer covering said adhesive surfaces prior to use;
g. a signal processor, formed of flexible circuitry, positioned underneath said electrically non-conductive adhesive surface and in electronic communication with said first and second electrodes;
h. a processor memory, formed of flexible circuitry, positioned underneath said electrically non-conductive adhesive surface and in electronic communication with said signal processor; and
i. a wireless transceiver, formed of flexible circuitry, positioned underneath said electrically non-conductive adhesive surface and in electronic communication with said signal processor and with said processor memory;
j. wherein said wireless transceiver is operable to receive commands to transmit one of:
i. real-time data from said signal processor; and
ii. stored data from said processor memory.

19. The system of claim 17, wherein said first computer application is hosted on said portable device that is operable to:
a. receive data from said EKG device via a wireless link;
b. host said computer application that is capable of creating at least one data product;
c. display said data and said at least one data product;
d. accept, store, and communicate user input; and
e. communicate, at least partially over a wireless link, with a website server.

20. The system of claim 19, wherein said web server is operable to:
a. communicate, at least partially over a wireless link, with said portable device hosting said first computer application;
b. host said second computer application that is operable to:
i. analyze said data;
ii. create at least one additional data product;
iii. format said data and said at least one additional data product for display;
iv. store said data, said at least one data product, and said at least one additional data product;
v. provide said data, said at least one data product, and said at least one additional data product for display; and
c. communicate said data, said at least one data product, and said at least one additional data product to a medical provider for analysis.
